(19) United States
(12) Patent Application Publication
(10) Pub. No.: US 2026/0102798 A1
(43) Pub. Date: Apr. 16, 2026

(54) METHOD FOR RESTORING AND MANAGING GARBAGE LANDFILL

(71) Applicant: POLAR BRIGHT ENVIRONMENTAL LIMITED, HONG KONG (CN)

(72) Inventors: ZHONG WANG, HONG KONG (CN); ZHI WANG, HONG KONG (CN)

(21) Appl. No.: 19/422,826

(22) Filed: Dec. 17, 2025

Related U.S. Application Data
(63) Continuation of application No. PCT/CN2025/120042, filed on Sep. 9, 2025.

(30) Foreign Application Priority Data
Sep. 12, 2024 (CN) 202411280925.6

(51) Int. Cl. B09B 3/60; A61L 9/14; B09B 3/30; B09B 3/40; C02F 1/44; C02F 103/06

(52) U.S. Cl. CPC B09B 3/60; A61L 9/14; B09B 3/30; B09B 3/40; C02F 1/441; C02F 2103/06

(57) ABSTRACT

A method for restoring and managing a garbage landfill is disclosed. The method comprises: determining a stabilization degree of each garbage dump within the landfill; performing a first garbage treatment step when the stabilization degree of the garbage dump meets a preset standard; performing aerobic stabilization pretreatment when the stabilization degree does not meet the preset standard, followed by a preset second garbage treatment step; performing ecological restoration treatment, so that a restored landfill meets a preset usage standard, wherein the first garbage treatment step is performed in an area adjacent to a garbage dump, and the second garbage treatment step is performed in an area where the garbage dump is located. By combining in-situ and ex-situ restoration, the method achieves harmless, reduced, and resource-based removal of garbage dumps while controlling ecological and environmental impacts, including leachate, odor, noise, and water and soil loss.

### CROSS-REFERENCES TO RELATED APPLICATIONS

The present disclosure is a continuation application of International application No. PCT/CN2025/120042, filed on Sep. 9, 2025, which claims priority to Chinese Patent Application No. 202411280925.6, filed on Sep. 12, 2024, with the Chinese Patent Office, the disclosures of which are incorporated herein by reference in their entireties.

### TECHNICAL FIELD

The present application relates to the technical field of garbage treatment, in particular to a method for restoring and managing a garbage landfill.

### BACKGROUND ART

A garbage landfill is a common waste treatment facility. It is a specific area and is equipped with corresponding facilities for collecting and dumping waste from municipal, industrial, and other sources after a series of operation links such as collection, transportation, dumping, compaction, and covering.

Restoration and management of a garbage landfill refer to a series of operational processes for restoring and managing an existing or abandoned garbage landfill to reduce environmental pollution and enhance the reusability value of land resources.

It typically integrates various technologies such as a land reclamation technology, a wastewater treatment technology, a gas control technology, a bioremediation technology, a geographic information system (GIS) technology, and a resource reuse technology to achieve the goals of reducing environmental pollution risks and enhancing the sustainable use value of land resources.

However, existing operations for restoring and managing a garbage landfill have defects such as high time and operating costs, low overall utilization levels, and inability to effectively eliminate ecological and environmental impacts.

### SUMMARY

The present application aims to provide a method for restoring and managing a garbage landfill, which is capable of solving at least some of the defects existing in traditional restoration of a garbage landfill.

In a first aspect, the embodiments of the present application provide the following technical solution: a method for restoring and managing a garbage landfill. The method includes: determining a stabilization degree of each garbage dump within the garbage landfill; performing a preset first garbage treatment step when the stabilization degree of the garbage dump meets a preset standard; performing aerobic stabilization pretreatment by introducing oxygen and microbial agents into the garbage dump when the stabilization degree of the garbage dump does not meet the preset standard; performing a preset second garbage treatment step after the aerobic stabilization pretreatment is completed; and performing ecological restoration treatment on the garbage landfill where garbage has been treated and on which the first garbage treatment step and/or the second garbage treatment step have/has been performed, so that a restored garbage landfill meets a preset usage standard, wherein the first garbage treatment step is performed in an area adjacent to the garbage dump, and the second garbage treatment step is performed in an area where the garbage dump is located.

Optionally, the second garbage treatment step includes: excavating the garbage dump under the protection of at least one preset protective measure; performing screening treatment on stockpiled garbage obtained by excavating the garbage dump to form multiple different types of garbage; and performing preset resource utilization treatment according to characteristics of the different types of garbage to form multiple types of recycled resources.

Optionally, the step of the aerobic stabilization pretreatment specifically includes: introducing oxygen into the garbage dumps through an air injection system and adding facultative microbial agents for stabilization treatment; and extracting air from an inside of the garbage dump through an air extraction system, wherein during the stabilization treatment, a plurality of different treatment indicators are monitored to determine a state of the garbage dump, and the air injection system and the air extraction system are controlled according to the state of the garbage dump; and wherein the treatment indicators include: pressure, flow rate, temperature, humidity, gas composition, and dump settlement, injection wells of the air injection system are arranged in an equidistant plum blossom shape or well shape, and the air extraction system is provided with an air filtration device for filtering air from an inside of the garbage dump.

Optionally, the protective measure includes: arranging a three-dimensional deodorization system to remove odor during the excavation of the garbage dump, wherein the three-dimensional deodorization system includes: an inflatable shed surrounding the garbage dump, an air deodorization device at an excavation working face, and a spray deodorization device located on a periphery of the inflatable shed.

Optionally, the second garbage treatment further includes: collecting leachate generated during the excavation of the garbage dump through a leachate collection system; and treating the leachate using a preset wastewater treatment process, wherein the preset wastewater treatment process specifically includes: performing pretreatment on the collected leachate to form homogeneous wastewater to be treated; treating the wastewater to be treated through a reverse osmosis concentration system to obtain wastewater that meets a discharge standard; and treating concentrate produced by the reverse osmosis concentration system using an evaporation crystallization technology.

Optionally, treating the wastewater to be treated through the reverse osmosis concentration system specifically includes: passing the wastewater to be treated through a two-stage DTRO system to form first effluent and first concentrate; storing the first concentrate in a concentrated water tank; passing the first concentrate stored in the concentrated water tank through a two-stage HPRO treatment system to form second effluent and second concentrate; merging the first effluent and the second effluent into a degassing tower for degassing treatment to obtain effluent after removing free carbon dioxide; and treating the effluent after removing free carbon dioxide through an ion exchanger to obtain the wastewater that meets the discharge standard; and treating the concentrate produced by the reverse osmosis concentration system using the evaporation crystallization technology specifically includes: performing evaporation treatment on the second concentrate through an evaporator to form crystalline salt and condensate; returning the condensate to an input end of the two-stage HPRO treatment system for reverse osmosis treatment again; and performing solidifying and drying treatment on the crystalline salt to form tailings, wherein the tailings can be used for backfilling the garbage landfill.

Optionally, performing screening treatment on the stockpiled garbage obtained by excavating the garbage dump specifically includes: placing the stockpiled garbage excavated from the garbage dump in a drying area for drying, and collecting leachate generated in the drying area through leachate collection; transporting dried stockpiled garbage to a first vibrating screen for sorting to sort out bricks, stones, and tiles with sizes larger than a preset first standard from the stockpiled garbage; sorting remaining stockpiled garbage after sorting through the first vibrating screen by a magnetic separator to sort out metals therefrom; and sorting remaining stockpiled garbage from which metals are sorted out through several roller screens and air separators, and sorting to form humus soil, bricks, stones, and tiles of various sizes, and light substances according to a preset sorting method.

Optionally, the drying area is a hardened concrete ground, to which a slope of 3‰ is set; a leachate drainage ditch is provided on a peripheral edge of the drying area, and the leachate drainage ditch is covered with a steel grating cover plate; and a surface water drainage ditch is additionally provided on a periphery of the leachate drainage ditch, wherein, when there is rainfall, the drying area is also covered with a removable double smooth-faced geotechnical membrane, a thickness of the double smooth-faced geotechnical membrane is 0.75 mm, and the double smooth-faced geotechnical membrane is removed when there is no rainfall.

Optionally, sorting remaining stockpiled garbage from which metals are sorted out through several roller screens and air separators, and sorting to form humus soil, bricks, stones, and tiles with sizes larger than a second standard, and light substances according to the preset sorting method specifically includes: sorting the remaining stockpiled garbage from which metals are sorted through a first roller screen for sorting to form an oversize material with a size larger than the second standard and an undersize material with a size smaller than the second standard; screening the oversize material with the size larger than the second standard through a first air separator, and sorting to form bricks, stones, and tiles with sizes between the second standard and the first standard, and light substances; sorting the undersize material with the size smaller than the second standard through a second roller screen to form an oversize material with a size larger than a third standard and an undersize material with a size smaller than the third standard; screening the oversize material with the size larger than the third standard through a second air separator, and sorting to form bricks, stones, and tiles with sizes between the third standard and the second standard, and light substances; sorting the undersize material with the size smaller than the third standard through a third roller screen to form an oversize material with a size larger than a fourth standard and humus soil with a size smaller than the fourth standard; and screening the oversize material with the size larger than the fourth standard through a second air separator, and sorting to form bricks, stones, and tiles with sizes between the fourth standard and the third standard, and light substances.

Optionally, performing preset resource utilization treatment according to characteristics of the different types of garbage to form multiple types of recycled resources specifically includes: performing firing treatment on the humus soil to form building materials or ceramsite; performing pyrolysis treatment on the light substances, and converting to obtain fuel oil, combustible gas, and carbon black.

The method for restoring and managing a garbage landfill provided by the embodiments of the present application can achieve the harmless, reduced, and resource-based removal of garbage dumps. During the restoring and managing process, the ecological and environmental impacts such as leachate, odor, noise, solid waste, soil erosion, etc. throughout the entire life cycle of restoring and managing a landfill are strictly controlled. Moreover, through the on-site excavation and classification treatment of the stockpiled garbage in the garbage landfill, the transportation distance and treatment cost of the garbage can be effectively reduced, the resource utilization level is high and economically reasonable, and the construction period can also be effectively shortened.

### BRIEF DESCRIPTION OF THE DRAWINGS

One or more embodiments are illustratively described through the pictures in the corresponding drawings. These illustrative descriptions do not constitute limitations to the embodiments. Elements with the same reference numbers in the drawings represent similar elements. Unless otherwise stated, the figures in the drawings are not to scale.

FIG. 1 is a method flowchart of a method for restoring and managing a garbage landfill provided by an embodiment of the present application;

FIG. 2 is a method flowchart of garbage treatment steps provided by an embodiment of the present application;

FIG. 3 is a schematic diagram of a wastewater treatment process provided by an embodiment of the present application;

FIG. 4 is a schematic diagram of screening treatment of stockpiled garbage provided by an embodiment of the present application; and

FIG. 5 is a schematic structural diagram of a drying area provided by an embodiment of the present application.

### DETAILED DESCRIPTION

To facilitate the understanding of the present application, the present application is described in more detail below with reference to the drawings and specific embodiments. It should be noted that when an element is described as being “fixed to” another element, it can be directly on another element or there can be one or more intervening elements therebetween. When an element is described as being “connected” to another element, it can be directly connected to another element or there can be one or more intervening elements therebetween. The orientations or positional relationships indicated by the terms “upper”, “lower”, “inner”, “outer”, “bottom”, etc. used in the specification are based on the orientations or positional relationships shown in the drawings, and are only for the convenience of describing the present application and simplifying the description, rather than indicating or implying that the referred device or element must have a specific orientation and be constructed and operated in a specific orientation, and therefore cannot be understood as limitations to the present application. Furthermore, the terms “first”, “second”, “third”, etc., are used for descriptive purposes only and cannot be understood as indicating or implying relative importance.

Unless otherwise defined, all technical and scientific terms used in the specification have the same meanings as commonly understood by those skilled in the technical field of the present application. The terms used in the specification of the present application are for the purpose of describing specific embodiments only and are not intended to limit the present application. The term “and/or” used in the specification includes any and all combinations of one or more related listed items.

Furthermore, the technical features involved in different embodiments of the present application described below can be combined with each other as long as they do not conflict with each other.

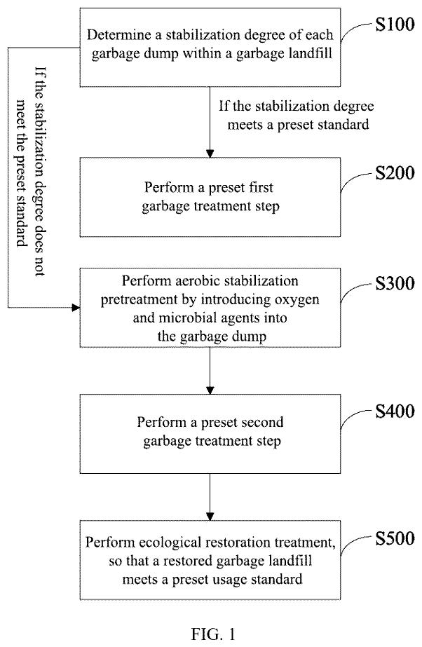

FIG. 1 shows a method for restoring and managing a garbage landfill provided by an embodiment of the present application. As shown in FIG. 1, the method for restoring and managing may include the following steps:
S100, determine a stabilization degree of each garbage dump within the garbage landfill.

Here, the “stabilization degree” refers to a series of standards or data indicators used for characterizing whether the garbage dump is in a stable state. A garbage dump with a high stabilization degree usually indicates that most of the organic matter in garbage has been decomposed, the generation of harmful gas has decreased, and the garbage volume is stable, etc.

Specifically, data parameters used for characterizing the stabilization degree may include, but are not limited to: organic matter content, temperature, gas production volume, and garbage volume stability, etc.
S200, perform a preset first garbage treatment step when the stabilization degree of the garbage dump meets a preset standard.

Here, the preset standard is a judgment standard pre-set by technical personnel according to actual needs, as long as it can distinguish whether different garbage dumps are stable, which is not specifically limited here.

The “first garbage treatment step” refers to a series of garbage treatment steps performed for stable garbage dumps. It can be performed in equipment workshops set up in areas adjacent to or near the garbage dumps, to realize the treatment and recycling of the stockpiled garbage within the garbage dumps.
S300, perform aerobic stabilization pretreatment by introducing oxygen and microbial agents into the garbage dumps when the stabilization degree of the garbage dumps does not meet the preset standard.

Here, if the preset standard is not met, it indicates that the garbage dumps are temporarily in an unstable state, and excavation and subsequent garbage treatment steps cannot be directly performed. Therefore, it can be appropriate to accelerate its transition to a suitable stable state through aerobic stabilization pretreatment by introducing oxygen and microbial agents.

Specifically, the step of the aerobic stabilization pretreatment specifically includes: introducing oxygen into the garbage dumps through an air injection system and adding facultative microbial agents for stabilization treatment; and extracting air inside the garbage dumps through an air extraction system to accelerate the stabilization degree of the garbage dumps.

During the stabilization treatment of the garbage dumps using the air injection system and the air extraction system, a plurality of different treatment indicators can be monitored to determine the state of the garbage dumps, and the air injection system and the air extraction system can be controlled based on the monitored state of the garbage dumps.

Here, the treatment indicators include, but are not limited to: pressure, flow rate, temperature, humidity, gas composition, and dump settlement. The injection wells of the air injection system can be arranged in an equidistant plum blossom or well shape, the diameter of each injection well can be controlled between 30 cm and 60 cm, and an interval between adjacent injection wells is 15 m. The air extraction system can also be provided with an air filtration device for filtering air from inside the garbage dump to ensure the cleanliness of the discharged air.

In some cases, even after the garbage landfill has been closed for many years and been out of use, the garbage dump may still have CO and H₂S gas exceeding personnel exposure limits and CH₄ gas content reaching explosion limits. The overall gas production of the entire garbage dump is still large, and the safety risks of toxic and harmful gas and flammable and explosive gas remain high, thereby being in an unstable state.

Therefore, the aforementioned aerobic stabilization pretreatment can accelerate the biodegradation process of the stockpiled garbage, change the internal environment of the garbage dump, quickly eliminate malodorous gas, reduce moisture, and reduce the methane concentration in the dump, thereby improving the stabilization degree of the garbage dump and providing a safe and reliable guarantee for the subsequent excavation and treatment of the stockpiled garbage.

Additionally, the aforementioned processes of alcoholization and acidification utilizing microbial fermentation can also convert liquid organic matter with larger molecular weights and higher viscosity in the garbage into alcohols and acids with smaller molecular weights, which are carried into the leachate for treatment.
S400, perform a preset second garbage treatment step after the aerobic stabilization pretreatment is completed.

Here, the “second garbage treatment step” also refers to a series of garbage treatment steps performed on the stockpiled garbage in the garbage dump. It can adopt the same or similar manner as the aforementioned first garbage treatment step for treatment to achieve the treatment and recycling of the stockpiled garbage within the garbage dump.
S500, perform ecological restoration treatment on the garbage landfill where the stockpiled garbage has been treated, so that the restored garbage landfill meets a preset usage standard.

Here, after the respective garbage dumps in the garbage landfill have been treated by the first garbage treatment step and the second garbage treatment step, and the stockpiled garbage is basically eliminated, subsequent ecological restoration treatment can be further performed so that the corresponding area can meet the preset usage standard.

The specific ecological restoration treatment performed can be set according to actual needs (e.g., the future land development and utilization plan for the landfill) to enable it to meet the requirements of the preset usage standard. For example, according to the requirements of “Technical Requirements for Stabilized Site Utilization for Household Garbage Landfill” (GB/T25179), the site can be ecologically restored to meet the technical requirements for medium to high-intensity utilization.

The method for restoring and managing provided in the embodiments of the present application classifies and treats garbage dumps, and ultimately restores their land values after completing the harmless removal and ecological restoration of the household garbage landfill.

On one hand, for the garbage dump with a high stabilization degree, a site is selected in peripheral vacant land in the landfill to build a sorting workshop, and the stockpiled garbage is further screened and then subjected to classified harmless, resource-based, and energy-based utilization.

On the other hand, for the garbage dump with a poor stabilization degree, when in-situ aerobic stabilization restoration is performed, and technical indicators such as methane and odor emissions and dump stability monitored reach the excavation conditions (i.e., when the stabilization degree meets the preset standard), the stockpiled garbage in the garbage landfill is excavated, screened, and subjected to resource utilization.

The following takes the second garbage treatment step as an example to describe in detail the specific processes of excavating and treating the stockpiled garbage of the garbage dump, as well as the technical effects that can be achieved. The second garbage treatment step described in one or more embodiments can be arbitrarily combined to achieve combinations of the technical effects.

It should be noted that the first garbage treatment step can adopt the same manner as the second garbage treatment step described in one or more embodiments and achieve similar garbage treatment effects. To avoid repetitive description, the first garbage treatment step will not be described in detail below.

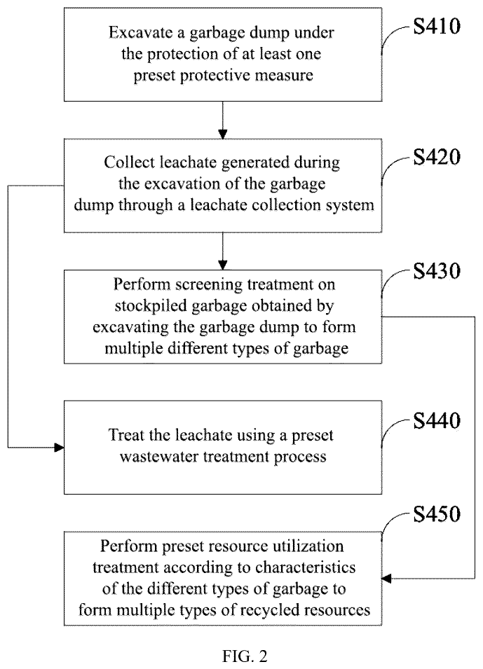

FIG. 2 is a schematic diagram of the second garbage treatment step provided by an embodiment of the present application. As shown in FIG. 2, the second garbage treatment step includes:
S410, excavate the garbage dump under the protection of at least one preset protective measure.

Here, “protective measures” refer to a series of measures adopted to avoid the environmental impact factors generated during the excavation of the garbage dump.

Specifically, the protective measures can include: arranging a three-dimensional deodorization system to remove odor during the excavation of the garbage dump. The three-dimensional deodorization system consists of an inflatable shed surrounding the garbage dump, an air deodorization device at an excavation working face, and a spray deodorization device located on the periphery of the inflatable shed.

In some other embodiments, the protective measures can also include: for equipment with high noise, taking noise reduction, sound insulation, and shock absorption measures, and using transport vehicles with low operating noise to reduce generated noise; and taking temporary barriers, drainage measures, covering, and other soil and water conservation prevention measures in building construction areas, roads and areas requiring hardening, and temporary occupation areas.
S430, perform screening treatment on stockpiled garbage obtained by excavating the garbage dump to form multiple different types of garbage.

Herein, “screening treatment” refers to the fact that one or more sorting devices are used for sorting the excavated stockpiled garbage based on sizes, thereby forming multiple different types and size standards of garbage to facilitate a series of subsequent treatment.

Preferably, the aforementioned screening treatment can be performed in simple enclosed workshops to prevent dust and odor from escaping and to meet the requirement for the low moisture content during garbage treatment in the screening system. The specifically-used screening system can be specifically configured according to actual needs (e.g., screening scale, working system, and process equipment), etc., and is not limited here.
S450, perform preset resource utilization treatment according to characteristics of the different types of garbage to form multiple types of recycled resources.

Here, “resource utilization treatment” refers to the conversion process where garbage is converted into recycled resources that can be recycled and reused through corresponding treatment based on its different characteristics. Different types of garbage can be converted into different recycled resources based on its own characteristics.

For example, the construction garbage (e.g., bricks, stones, tiles) obtained by sorting can be mixed with humus soil for producing new building materials and firing ceramsite after harmless treatment, or used for manufacturing permeable bricks for urban roads or for on-site backfilling in the garbage landfill. The sorted light substances such as waste plastic and sediment can be regenerated and utilized through pyrolysis processes.

In some embodiments, for the leachate generated during the excavation of the garbage dump, the second garbage treatment further includes an additional leachate treatment step. Continue to refer to FIG. 2, the second garbage treatment further includes:
S420, collect leachate generated during the excavation of the garbage dump through a leachate collection system.
S440, treat the leachate using a preset wastewater treatment process.

Here, the preset wastewater treatment process specifically includes: first, performing pretreatment on collected leachate to form homogeneous wastewater to be treated; then, treating the wastewater to be treated through a reverse osmosis concentration system to obtain wastewater that meets a discharge standard; and finally, treating concentrate produced by the reverse osmosis concentration system using an evaporation crystallization technology.

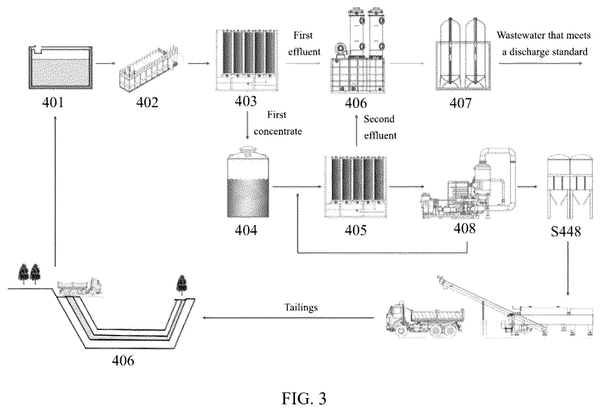

In some embodiments, FIG. 3 is a schematic diagram of the wastewater treatment process provided by an embodiment of the present application. As shown in FIG. 3, the wastewater treatment process may specifically include the following steps:
S441, concentrating leachate obtained from collection into a regulating tank 401 for temporary storage, wherein the regulating tank 401 serves to regulate water quality and quantity, remove suspended solids, and regulate wastewater flow rate, etc.;
S442, subjecting the leachate output from the regulating tank 401 to one or more pretreatment operations through a pretreatment module 402, wherein these pretreatment operations include solid separation (e.g., preliminary separation and removal of large solid particles, suspended solids, and sediment from the wastewater), water quality regulation (e.g., regulating pH, removing some organic matter from wastewater, etc.) and other operations.
S443, passing the wastewater to be treated through a two-stage DTRO treatment system 403 to form first effluent and first concentrate, wherein the first concentrate is stored in a concentrated water tank 404;
S444, passing the first concentrate stored in the concentrated water tank 404 through a two-stage HPRO treatment system 405 to form second effluent and second concentrate;
S445, merging the first effluent and the second effluent into a degassing tower 406 for degassing treatment to obtain effluent after removing free carbon dioxide;
S446, treating the effluent after removing free carbon dioxide through an ion exchanger 407 to obtain wastewater that meets the discharge standard;
S447, performing evaporation treatment on the second concentrate through an evaporator 408 to form crystalline salt and condensate;
S448, returning the condensate to an input end of the two-stage HPRO treatment system 405 for reverse osmosis treatment again; and
S449, performing solidifying and drying treatment on the crystalline salt to form tailings, and transporting the tailings to the garbage landfill 406 via transport vehicles for backfilling.

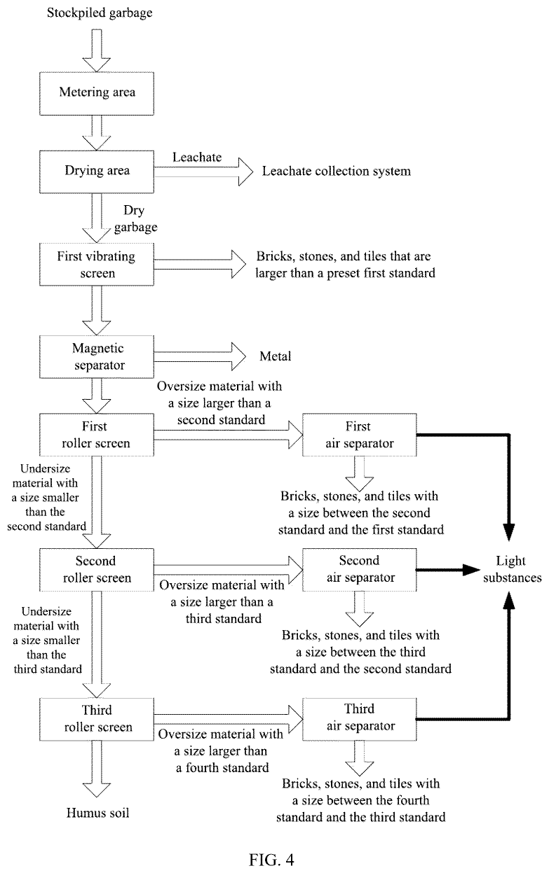

In some embodiments, FIG. 4 is a schematic diagram of the screening treatment provided by an embodiment of the present application. As shown in FIG. 4, the screening treatment may include the following steps:
S431, place the stockpiled garbage excavated from the garbage dump in a drying area for drying.

Here, the leachate generated in the drying area is associated with the aforementioned step S420, and the leachate generated during the drying process is collected by the leachate collection system, and is further provided to the wastewater treatment process shown in FIG. 3 for treatment.

Specifically, the stockpiled garbage obtained during excavation can first be measured in a metering area to determine the volume and weight before being moved to the drying area for drying, so as to facilitate accurate control and regulation of the excavation progress.

Additionally, the area of the drying area can be set according to actual needs. For example, it can be set based on the stockpiled garbage treated daily. The time required for drying can also be set according to actual needs. For example, it can be set to be 2 days to 7 days.

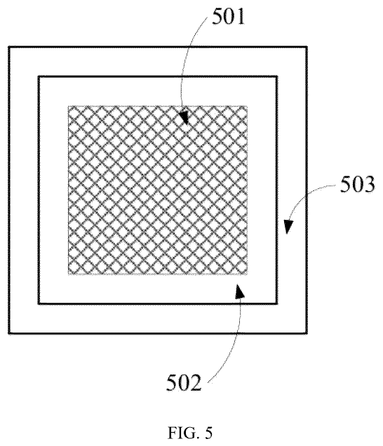

In some embodiments, in order to ensure the collection effect of leachate, the drying area can be appropriately set up and configured with a suitable and compatible matching device. As shown in FIG. 5, the collection device may include: a drying area 501, a leachate drainage ditch 502, and a surface water drainage ditch 503.

Here, the leachate drainage ditch 502 is provided on the peripheral edge of the drying area 501 for collecting and guiding leachate. Its size is 0.3 m×0.3 m, and the upper surface is covered with a steel grating cover plate. The drying area 501 is a hardened concrete ground, to which a slope of 3‰ is set to facilitate the flow of leachate under gravity to the leachate drainage ditch 502 on the peripheral edge. The surface water drainage ditch 503 is provided on the periphery of the leachate drainage ditch 502 and can be used for guiding and draining rainwater that flows into the drying area and its surroundings.

Preferably, the drying area 501 can be configured with an HDPE double smooth-faced geotechnical membrane having a thickness of 0.75 mm. When there is rainfall, the double smooth-faced geotechnical membrane is temporarily covered over the drying area 501 and the leachate drainage ditch 502, thereby controlling generation and pollution of the leachate, reducing the impact of rainwater on the garbage dump, and protecting the environment against potential adverse effects caused by restoring and managing the garbage landfill.
S432, transport dried stockpiled garbage to a first vibrating screen for sorting to sort out bricks, stones, and tiles with sizes larger than a preset first standard from the stockpiled garbage.
S433, sort remaining stockpiled garbage after sorting through the first vibrating screen by a magnetic separator to sort out metals therefrom.
S434, sort remaining stockpiled garbage from which metals are sorted out through several roller screens and air separators, and sort to form humus soil, bricks, stones, and tiles of various sizes, and light substances according to a preset sorting method.

Specifically, the aforementioned step S434 can use a three-stage screening manner, use devices with three different screening sizes, and further sort out three different sizes of bricks, stones, and tiles from the remaining stockpiled garbage of step S433. Continue to refer to FIG. 4. The step S433 specifically includes:
S4341, sorting the remaining stockpiled garbage after sorting out the metals through a first roller screen to form an oversize material with a size larger than the second standard and an undersize material with a size smaller than the second standard;
S4342, screening the oversize material with the size larger than the second standard through a first air separator, and sorting to form bricks, stones, and tiles with sizes between the second standard and the first standard, and light substances;
S4343, sorting the undersize material with the size smaller than the second standard through a second roller screen to form an oversize material with a size larger than a third standard and an undersize material with a size smaller than the third standard;
S4344, screening the oversize material with the size larger than the third standard through a second air separator, and sorting to form bricks, stones, and tiles with sizes between the third standard and the second standard, and light substances;
S4345, sorting the undersize material with the size smaller than the third standard through a third roller screen to form an oversize material with a size larger than a fourth standard and humus soil with a size smaller than the fourth standard; and
S4346, screening the oversize material with the size larger than the fourth standard through a second air separator, and sorting to form bricks, stones, and tiles with sizes between the fourth standard and the third standard, and light substances.

Thus, through the above series of steps, the dried stockpiled garbage can be further sorted into: humus soil, bricks/stones/tiles with sizes larger than the first standard, bricks/stones/tiles with sizes between the second standard and the first standard, bricks/stones/tiles with sizes between the third standard and the second standard, bricks/stones/tiles with sizes between the fourth standard and the third standard, and light substances.

Specifically, the aforementioned first standard is 200 mm, the second standard is 70 mm, the third standard is 40 mm, and the fourth standard is 10 mm.

In some embodiments, the humus soil sorted out from the garbage landfill is rich in organic matter, and the content of components such as SiO₂ and Al₂O₃ is also relatively high. Based on these characteristics, a corresponding building material integration system can be used for performing cooperative treatment on humus soil, construction garbage, along with solid wastes such as waste shale and sludge, to produce building materials (fired bricks, non-fired bricks) and fired ceramsite.

Additionally, the resource utilization of light substances such as plastic in the stockpiled garbage in the landfill is affected by high impurity content in the raw materials, and continuous low-temperature pyrolysis treatment can be used to convert low-quality waste plastic into fuel oil, combustible gas, and carbon black. The fuel oil and combustible gas can be stored or sold, while carbon black is relatively stable and easy to store, and can be used as general fuel.

Preferably, these pyrolysis products (e.g., combustible gas and carbon black) can be further used as a heat source in the aforementioned firing process of humus soil, thereby reducing or eliminating reliance on external energy.

According to the method for restoring and managing provided by the embodiments of the present application, on-site treatment is performed on the stockpiled garbage in the landfill to form resource utilization. After the sorting treatment, the sorted humus soil is made into new building materials (fired bricks) or sintered into ceramsite (used for landscaping or as building fill); and the light substances (waste plastic) are subjected to low-temperature pyrolysis to produce oil, gas, and carbon black, which are further used as energy in the resource production process of humus soil. Therefore, the time cost for outward transport of humus soil and the like can be greatly reduced, operating costs can be lowered, and better environmental benefits, social benefits, and economic benefits can be obtained.

In summary, according to the method for restoring and managing provided by the embodiments of the present application, the resource utilization level of the project can be significantly improved through the resource utilization of stockpiled garbage. On one hand, the economic benefits of humus soil resource utilization are improved. On-site resourcing of humus soil is adopted for supplying building materials and greening needed for surrounding development, especially ceramsite; and as the humus soil has been harmless after high-temperature sintering, it can be sold to the market, increasing the project's economic returns. On the other hand, the economic benefits of resource utilization of the light oversize material are improved. The light oversize material is subjected to a thermal pyrolysis process to produce fuel oil as well as combustible gas and carbon black. The fuel oil can be directly sold, and the combustible gas and carbon black are used as thermal fuel for the deep processing of humus soil, reducing the use of external energy and increasing project revenue.

Moreover, the entire process for restoring and managing follows the principles of “harmlessness, reduction, and resource utilization”, enabling the excavation and disposal of the garbage landfill to truly form two cores of “technical closed-loop” and “economic closed-loop”. There is no need to transport humus soil to other places for landfilling, which can completely solve the environmental safety hazards of the garbage landfill, effectively control the impact of garbage on the ecological environment, and significantly reduce the impact of historical stockpiled garbage on the areas surrounding the landfill. Through the resource and energy utilization of stockpiled garbage, the carbon emissions of the garbage landfill can be effectively reduced, making a positive contribution to achieving the goals of pollution reduction and carbon reduction.

Finally, it should be noted that the above embodiments are only used to illustrate the technical solutions of the present application, but not to limit them; under the concept of the present application, the technical features in the above embodiments or different embodiments can also be combined, steps can be implemented in any order, and there are many other variations of the different aspects of the present application as described above. For simplicity, they are not provided in detail; although the present application has been described in detail with reference to the foregoing embodiments, those of ordinary skill in the art should understand that they can still modify the technical solutions described in the foregoing embodiments or equivalently replace some of the technical features; and these modifications or replacements do not make the essence of the corresponding technical solutions deviate from the scope of the technical solutions of the embodiments of the present application.

What is claimed is:

1. A method for restoring and managing a garbage landfill, comprising:
determining a stabilization degree of each garbage dump within the garbage landfill;
performing a preset first garbage treatment step when the stabilization degree of the garbage dump meets a preset standard;
performing aerobic stabilization pretreatment by introducing oxygen and microbial agents into the garbage dump when the stabilization degree of the garbage dump does not meet the preset standard; and
performing a preset second garbage treatment step after the aerobic stabilization pretreatment is completed; and
performing ecological restoration treatment on the garbage landfill where garbage has been treated and on which the first garbage treatment step and/or the second garbage treatment step have/has been performed, so that a restored garbage landfill meets a preset usage standard,
wherein the first garbage treatment step is performed in an area adjacent to the garbage dump, and the second garbage treatment step is performed in an area where the garbage dump is located.

2. The method according to claim 1, wherein the second garbage treatment step comprises:
excavating the garbage dump under the protection of at least one preset protective measure;
performing screening treatment on stockpiled garbage obtained by excavating the garbage dump to form multiple different types of garbage; and
performing preset resource utilization treatment according to characteristics of the different types of garbage to form multiple types of recycled resources.

3. The method according to claim 1, wherein the steps of the aerobic stabilization pretreatment specifically comprise:
introducing oxygen into the garbage dump through an air injection system and adding facultative microbial agents for stabilization treatment; and extracting air from an inside of the garbage dump through an air extraction system, wherein
during the stabilization treatment, a plurality of different treatment indicators are monitored to determine a state of the garbage dump, and the air injection system and the air extraction system are controlled according to the state of the garbage dump; and
wherein the treatment indicators comprise: pressure, flow rate, temperature, humidity, gas composition, and dump settlement,
injection wells of the air injection system are arranged in an equidistant plum blossom shape or well shape, and the air extraction system is provided with an air filtration device for filtering air from an inside of the garbage dump.

4. The method according to claim 2, wherein the protective measures comprise:
arranging a three-dimensional deodorization system to remove odor during the excavation of the garbage dump,
wherein the three-dimensional deodorization system comprises: an inflatable shed surrounding the garbage dump, an air deodorization device at an excavation working face, and a spray deodorization device located on a periphery of the inflatable shed.

5. The method according to claim 1, wherein the second garbage treatment step further comprises:
collecting leachate generated during the excavation of the garbage dump through a leachate collection system; and
treating the leachate using a preset wastewater treatment process,
wherein the preset wastewater treatment process specifically comprises:
performing pretreatment on collected leachate to form homogeneous wastewater to be treated;
treating the wastewater to be treated through a reverse osmosis concentration system to obtain wastewater that meets a discharge standard; and
treating concentrate produced by the reverse osmosis concentration system using an evaporation crystallization technology.

6. The method according to claim 5, wherein the step of treating the wastewater to be treated through the reverse osmosis concentration system specifically comprises:
passing the wastewater to be treated through a two-stage DTRO treatment system to form first effluent and first concentrate;
storing the first concentrate in a concentrated water tank;
passing the first concentrate stored in the concentrated water tank through a two-stage HPRO treatment system to form second effluent and second concentrate;
merging the first effluent and the second effluent into a degassing tower for degassing treatment to obtain effluent after removing free carbon dioxide; and
treating the effluent after removing free carbon dioxide through an ion exchanger to obtain the wastewater that meets the discharge standard; and
the step of treating the concentrate produced by the reverse osmosis concentration system using the evaporation crystallization technology specifically comprises:
performing evaporation treatment on the second concentrate through an evaporator to form crystalline salt and condensate;
returning the condensate to an input end of the two-stage HPRO treatment system for reverse osmosis treatment again; and
performing solidifying and drying treatment on the crystalline salt to form tailings, wherein the tailings can be used for backfilling the garbage landfill.

7. The method according to claim 5, wherein the step of performing screening treatment on the stockpiled garbage obtained by excavating the garbage dump specifically comprises:
placing the stockpiled garbage excavated from the garbage dump in a drying area for drying, and collecting leachate generated in the drying area through leachate collection;
transporting dried stockpiled garbage to a first vibrating screen for sorting to sort out bricks, stones, and tiles with sizes larger than a preset first standard from the stockpiled garbage;
sorting remaining stockpiled garbage after sorting through the first vibrating screen by a magnetic separator to sort out metals therefrom; and
sorting remaining stockpiled garbage from which metals are sorted out through several roller screens and air separators, and sorting to form humus soil, bricks, stones, and tiles of various sizes, and light substances according to a preset sorting method.

8. The method according to claim 7, wherein the drying area is a hardened concrete ground, to which a slope of 3‰ is set;
a leachate drainage ditch is provided on a peripheral edge of the drying area, and the leachate drainage ditch is covered with a steel grating cover plate; and a surface water drainage ditch is additionally provided on a periphery of the leachate drainage ditch,
wherein, when there is rainfall, the drying area is also covered with a removable double smooth-faced geotechnical membrane, a thickness of the double smooth-faced geotechnical membrane is 0.75 mm, and the double smooth-faced geotechnical membrane is removed when there is no rainfall.

9. The method according to claim 7, wherein the steps of sorting remaining stockpiled garbage from which metals are sorted out through several roller screens and air separators, and sorting to form humus soil, bricks, stones, and tiles with sizes larger than a second standard, and light substances according to a preset sorting method specifically comprise:
sorting the remaining stockpiled garbage from which metals are sorted out through a first roller screen to form an oversize material with a size larger than the second standard and an undersize material with a size smaller than the second standard;
screening the oversize material with the size larger than the second standard through a first air separator, and sorting to form bricks, stones, and tiles with sizes between the second standard and the first standard, and light substances;
sorting the undersize material with the size smaller than the second standard through a second roller screen to form an oversize material with a size larger than a third standard and an undersize material with a size smaller than the third standard;
screening the oversize material with the size larger than the third standard through a second air separator, and sorting to form bricks, stones, and tiles with sizes between the third standard and the second standard, and light substances;
sorting the undersize material with the size smaller than the third standard through a third roller screen to form an oversize material with a size larger than a fourth standard and humus soil with a size smaller than the fourth standard; and
screening the oversize material with the size larger than the fourth standard through a second air separator, and sorting to form bricks, stones, and tiles with sizes between the fourth standard and the third standard, and light substances.

10. The method according to claim 9, wherein the step of performing preset resource utilization treatment according to characteristics of the different types of garbage to form multiple types of recycled resources specifically comprises:
performing firing treatment on the humus soil to form building materials or ceramsite; and
performing pyrolysis treatment on the light substances, and converting to obtain fuel oil, combustible gas, and carbon black.
